(19) United States
(12) Patent Application Publication
(10) Pub. No.: US 2023/0123308 A1
(43) Pub. Date: Apr. 20, 2023

(54) SYSTEM AND METHOD FOR CONFIGURABLE FLOW CONTROLLER

(71) Applicant: REPRO-MED SYSTEMS, INC., Chester, NY (US)

(72) Inventor: PETER TADDEO, Yonkers, NY (US)

(21) Appl. No.: 17/502,351

(22) Filed: Oct. 15, 2021

(51) Int. Cl. A61M 5/168; A61M 5/162; A61M 39/24

(52) U.S. Cl. CPC A61M 5/16881; A61M 5/16813; A61M 5/162; A61M 39/24

(57) ABSTRACT

Provided is a configurable flow controller for providing a selected known flow rate for a given pressure for the delivery of a liquid from a reservoir to a patient. The configurable flow controller includes a modular housing providing an inlet port and an outlet port for connection to tubing. A user configurable flow path is disposed within the modular housing between the inlet port and the outlet port defined by a plurality of optional pathway segments including at least one fixed-geometry passive check valve, each optional pathway segment having a predetermined resistance to flow, a selected subset of the optional pathway segments establishing the selected known flow rate for the liquid passing from the outlet port. A kit and method of use are also provided.

### FIELD OF THE INVENTION

The present invention relates generally to systems and methods for liquid fluid flow regulation as may be desired for the delivery of liquid for infusion to a patient, and more specifically to an improved flow controller incorporating fixed geometry flow path segments that may be aligned in varying configurations to achieve different flow rates.

### BACKGROUND

Infusion systems for the delivery of liquid pharmaceuticals are widely used and relied upon by patients and care givers alike. Such delivery is generally made in one of two ways. The first is an immediate delivery from a health care provider or other operator in the form of a simple injection performed with a syringe and a needle directly disposed to the tissue of the patient. For this type of immediate delivery, the amount of the pharmaceutical is typically measured by the health care provider or other operator and the rate of delivery is typically based on the speed at which they depress the plunger. Although overmedication can occur, the rate of delivery is rarely an issue with immediate delivery.

The second option is for gradual delivery, wherein a syringe or other reservoir is connected to specific medical tubing for delivery over time. With such time-based delivery, overmedication and/or overdose of the pharmaceutical is a very real possibility. Syringes, or other pharmaceutical reservoirs such as fluid bags, are typically easily and commonly adapted for use with many different types of pharmaceuticals, however the flow rate for proper delivery of such pharmaceuticals as determined by the manufacturer may vary widely. Further, as patient needs and situations are often different, even when dealing with the same type of pharmaceutical it may be necessary for different patients to receive different flow rates, which again would be at or below the manufacturer's specified maximum delivery rate.

With the ever-increasing desire to reduce health care costs, there is a market demand to reduce the costs of providing intravenous and subcutaneous administrations. With infusion over time, there are essentially two broad categories of system—one is constant flow/variable pressure, and the second is variable flow/constant pressure.

The goal of both systems is to provide a set volume of fluid over a time into a patient via a tubing and needle set. The flow rate of any fluid moving through a flow path is determined by the pressure gradient across the path and the resistance of the fluid through the path. This relationship is represented by the equation: Q=ΔP/R.

With the first system—constant flow/variable pressure—programmable pumps are used to control the rate of flow. These systems employ programmable pumps and attempt to accurately measure the flow rate and then algorithmically adjust the pressure to maintain the correct flow rate.

Because these systems attempt to maintain the same flow rate regardless of pressure, these systems generally incorporate a warning system to alert the user and/or operator of any dangerous increase in pressure as the pump attempts to maintain that constant flow. If there is an occlusion at the sight of administration, even with an alarm the patient may be injured and/or receive an overdose of the pharmaceutical.

In contrast to constant flow pumps, the second option of variable flow/constant pressure pump systems have been found to be safer and are often more financially acceptable to users. Variable flow/constant pressure systems have two relatively simpler challenges when compared to constant flow/variable pressure systems—first they must create a constant pressure gradient and second, they strive to maintain a precise resistance to ensure the intended flow rate.

Presently, resistance is generally controlled by infusion through tubing and needle sets. The tubing and needle sets, as well as the connectors may all be understood with the simplified view that they are all just tube elements of varying diameters and lengths.

The resistance of a tube flow path is determined by the length, viscosity of the fluid moving through the system divided by the radius of the tubing. This relationship is represented by the equation: R=8Lη/πr\hat{ }4. It should be noted that the overwhelming determining factor for resistance is the radius.

Standard medical tubing such as general IV tubing, has an internal diameter, or more specifically an internal radius “r” of such a great size that for normal and practical uses it effectively provides an unrestricted flow rate. More specifically, under Poiseuille's Law, the length of such tubing and internal radius are of such a size that standard tubing or IV tubing effectively provides no meaningful reduction in flow rate, especially when compared with the maximum dosage flow rate. While such general tubing may indeed govern flow rate when provided in lengths of 10's or 100's of meters, these lengths are impractical for normal use.

Infusion systems, and most specifically variable flow/constant pressure pump systems therefore utilize flow control tubing that has been specifically manufactured to provide very specific and constantly maintained internal diameters, and more specifically internal radius “r”.

As will be appreciated from the equation above, the overwhelming determining factor for resistance is the radius “r”. In other words, any variability in the radius during manufacturing of the flow control tubing will cause great variability in the product. This is problematic because as flow control tubing must be flexible it must be made from plastics which are difficult to control with respect to the formation of consistent inner diameters without great cost to achieve high tolerances for manufacturing.

Such tubing must also be carefully inspected and potentially reworked for re-calibration and the identification of very specific lengths carefully noted so as to consistently provide known flow rates for known pressures.

These elements are costly to achieve, and even a simple error or inconsistency may render a batch of tubing defective. Further, even when the internal diameter is very consistent, an inadvertent change in the length can have unintended consequences of increasing or decreasing the desired flow rate for a given pressure.

Hence there is a need for a method and system that is capable of overcoming one or more of the above identified challenges.

### SUMMARY OF THE INVENTION

Our invention solves the problems of the prior art by providing a novel configurable flow controller, kit and method of use therefore.

In particular, and by way of example only, according to one embodiment of the present invention, provided is a configurable flow controller for providing a selected known flow rate for a given pressure for the delivery of a liquid from a reservoir to a patient, including: a modular housing providing an inlet port structured and arranged for connection to a first tubing line from the reservoir and an outlet port structured and arranged for connection to a second tubing line to the patient; and a user configurable flow path disposed within the modular housing between the inlet port and the outlet port defined by a plurality of optional pathway segments including at least one fixed-geometry passive check valve, each optional pathway segment having a predetermined resistance to flow, a selected subset of the optional pathway segments establishing the selected known flow rate for the liquid passing from the outlet port.

For yet another embodiment, provided is a configurable flow controller for providing a selected known flow rate for a given pressure for the delivery of a liquid from a reservoir to a patient, including: a modular housing providing an inlet port structured and arranged for connection to a first tubing line from the reservoir and an outlet port structured and arranged for connection to a second tubing line to the patient; and a configurable flow path provided by at least a first flow rate regulator and at least a second flow rate regulator, each flow rate regulator providing selectable flow pathway segments including at least one fixed-geometry passive check valve and at least one through hole with each pathway segment having a predetermined resistance to flow, the selectable alignment of the first flow rate regulator to the second flow rate regulator aligning at least two flow pathway segments between the inlet port and the outlet port to provide the selected known flow rate for the liquid passing from the outlet port.

In yet another embodiment, provided is a configurable flow controller for providing a selected known flow rate for a given pressure for the delivery of a liquid from a reservoir to a patient, including: a segmented housing providing an inlet port structured and arranged for connection to a first tubing line from the reservoir and an outlet port structured and arranged for connection to a second tubing line to the patient; and at least two flow rate regulators disposed within the segmented housing, each flow rate regulator providing at least one selectable flow pathway segment with at least one selectable flow pathway segment being a fixed-geometry passive check valve; wherein a user selectable alignment of a first flow rate regulator to a second flow rate regulator aligns at least two flow pathway segments between the inlet port and the outlet port to provide the selected known flow rate for the liquid passing from the outlet port.

Still for yet another embodiment, provided is a kit for a configurable flow controller for providing a selected known rate for a given pressure rate for the delivery of a liquid from a reservoir to a patient, including: a segmented housing providing an inlet port structured and arranged for connection to a first tubing line from the reservoir and an outlet port structured and arranged for connection to a second tubing line to the patient; and a plurality of flow rate regulators to be disposed by a user within the segmented housing, each flow rate regulator providing at least one selectable flow path segment with at least one selectable flow path segment being a fixed-geometry passive check valve; wherein a user selectable alignment of at least a first flow rate regulator to a second flow rate regulator aligning at least two flow pathway segments between the inlet port and the outlet port as a configurable flow path to provide the selected known flow rate for the liquid passing from the outlet port.

And, yet still further, for yet another embodiment, provided is a method for using a configurable flow controller for providing a selected known flow rate for a given pressure for the delivery of a liquid from a reservoir to a patient, including: providing a modular housing having an inlet port structured and arranged for connection to a first tubing line from the reservoir and an outlet port structured and arranged for connection to a second tubing line to the patient; and providing at least two flow rate regulators to be disposed within the modular housing, each flow rate regulator providing at least one selectable flow pathway segment with at least one selectable flow pathway segment being a fixed-geometry passive check valve; selectively aligning a first flow rate regulator to a second flow rate regulator and disposing them within the modular housing to align and dispose at least two flow pathway segments between the inlet port and the outlet port as a configurable flow path to provide the selected known flow rate for the liquid passing from the outlet port; engaging tubing from the inlet port to the liquid reservoir; and engaging tubing from the outlet port to a needle set for the delivery of the liquid to the patient.

### BRIEF DESCRIPTION OF THE DRAWINGS

FIG. 1A is a side view illustration of a configurable flow controller in accordance with at least one embodiment;

FIG. 1B is a front perspective view of the configurable flow controller shown in FIG. 1A;

FIG. 1C is an exploded perspective view of the configurable flow controller shown in FIG. 1B;

FIG. 1D is a front plane view of a flow rate regulator as shown in FIG. 1C, further illustrating the selectable flow pathway segment as a fixed-geometry passive check valve pathway segment and a through hole;

FIG. 2 is an illustration of six different configurable flow path options permitted by the flow controller as shown in FIG. 1A-1C in accordance with at least one embodiment;

FIG. 3A is an enlarged illustration of a first flow path configuration provided by two flow rate regulators in accordance with at least one embodiment;

FIG. 3B is an enlarged illustration of a second flow path configuration provided by two flow rate regulators in accordance with at least one embodiment;

FIG. 3C is an enlarged illustration of a third flow path configuration provided by two flow rate regulators in accordance with at least one embodiment;

FIG. 3D is an enlarged illustration of a fourth flow path configuration provided by two flow rate regulators in accordance with at least one embodiment;

FIG. 3E is an enlarged illustration of a fifth flow path configuration provided by two flow rate regulators in accordance with at least one embodiment;

FIG. 3F is an enlarged illustration of a sixth flow path configuration provided by two flow rate regulators in accordance with at least one embodiment;

FIG. 4 is a perspective exploded view of a configurable flow controller incorporating the configured flow path shown in FIG. 3A;

FIG. 5 is a perspective exploded view of a configurable flow controller incorporating the configured flow path shown in FIG. 3C;

FIG. 6 is a perspective exploded view of a configurable flow controller incorporating the configured flow path shown in FIG. 3E;

FIG. 7 is a perspective front view of an assembled configurable flow controller incorporating the configured flow path shown in FIG. 3E and FIG. 6;

FIG. 8 is a perspective exploded view of an alternative configurable flow controller incorporating six flow rate regulators in accordance with at least one embodiment;

FIG. 9 is a conceptual illustration of a kit for providing a configurable flow controller in accordance with at least one embodiment;

FIG. 10 is a conceptual illustration of an infusion system incorporating a configurable flow controller in accordance with at least one embodiment; and

FIG. 11 is a high-level flow diagram of a method of providing a configurable flow controller in accordance with at least one embodiment.

### DETAILED DESCRIPTION

Before proceeding with the detailed description, it is to be appreciated that the present teaching is by way of example only, not by limitation. The concepts herein are not limited to use or application with a specific system or method for providing a certificate, and more specifically a certificate for network access. Thus, although the instrumentalities described herein are for the convenience of explanation shown and described with respect to exemplary embodiments, it will be understood and appreciated that the principles herein may be applied equally in other types of precision variable flow rate infusion systems and methods.

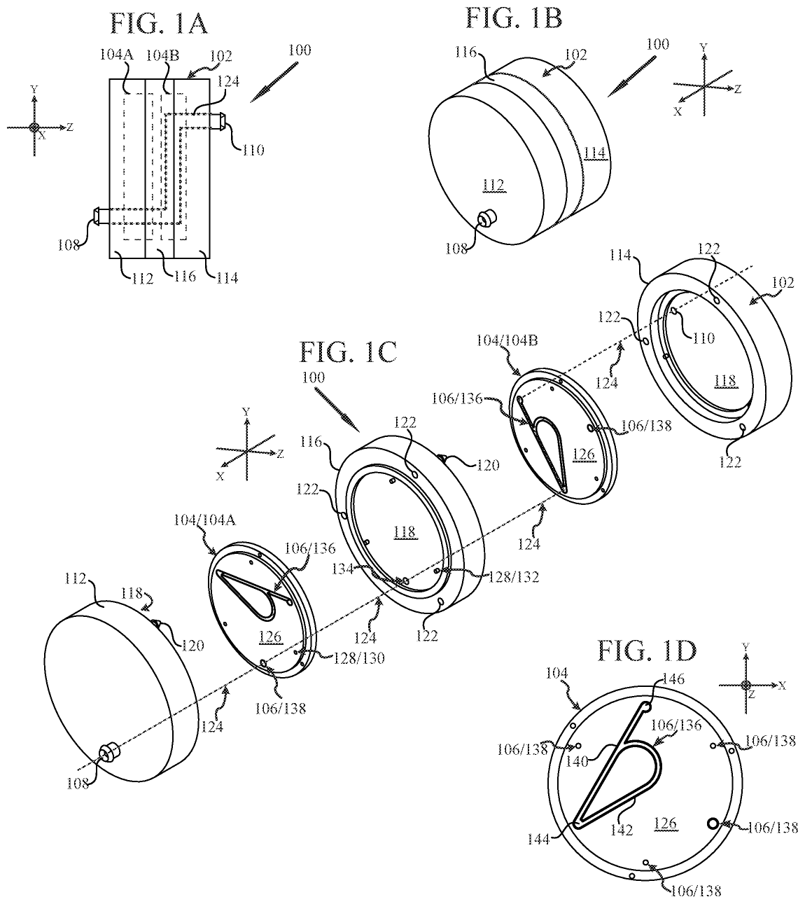

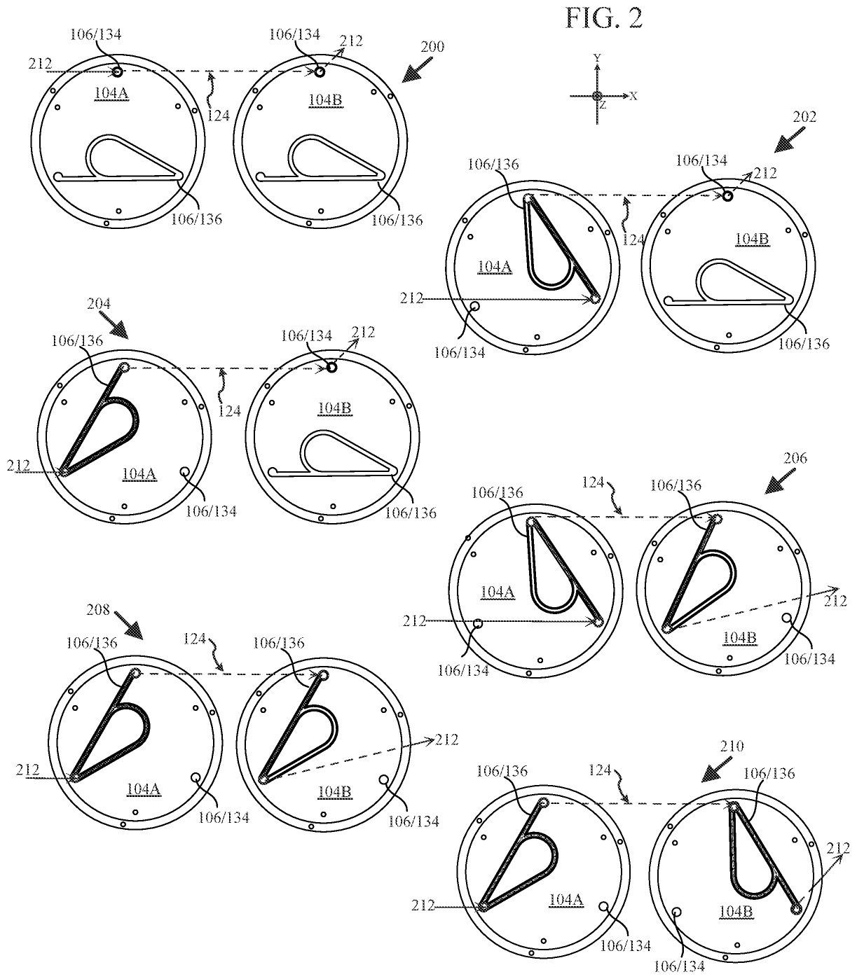

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 appears in FIG. 1.

Turning now to the drawings, and more specifically FIG. 1A through 1C, there is shown a Configurable Flow Controller, hereinafter CFC 100, according to at least one embodiment in side view (FIG. 1A) and front perspective view (FIG. 1B). CFC 100 is understood and appreciated as an advantageous component for flow rate control in an infusion system for delivering a liquid from a reservoir, having an initial potential outflow rate, to a patient.

To facilitate the description of systems and methods for embodiments of CFC 100, the orientation of CFC 100 as presented in the figures is referenced to the coordinate system with three axes orthogonal to one another as shown in FIG. 1. The axes intersect mutually at the origin of the coordinate system, which is chosen to be the center of CFC 100, however the axes shown in all figures are offset from their actual locations for clarity and ease of illustration.

For ease of discussion and illustration, FIG. 1C presents a perspective exploded view of CFC 100 as shown in FIGS. 1A and 1B. Moreover, CFC 100 is a modular device which may be quickly and easily assembled. With respect to FIGS. 1A-1C as shown, it may be appreciated that for at least one embodiment, CFC 100 comprises a segmented, or modular housing 102 and at least one flow rate regulator 104, e.g., a first flow rate regulator 104A and a second flow rate regulator 104B, that are disposed therein, each flow rate regulator 104 providing at least two selectable flow pathway segments 106. As is further set forth below, each selectable flow pathway segment 106 has a predetermined resistance to flow, or rather has been structured and arranged with a specific geometry to achieve a predetermined resistance to the flow of liquid therethrough.

The housing 102 provides an inlet port 108 and an outlet port 110. For at least one embodiment the housing 102 is provided as first end component 112 providing the inlet port 108 and as second end component 114 providing the outlet port 110, and at least one optional midsection component 116.

For at least one embodiment the first end component 112 and the second end component 114 may be substantially identical and therefore interchangeable, such that one manufactured element can serve as either the first end component 112 or the second end component 114. Alternatively due to varying embodiment of engagers to secure the modular housing 102, the first end component 112 and second end component 114 may still be interchangeable with respect to how an embodiment of CFC 100 is assembled, but each component is slightly different so as to accommodate different engagers/attachers/fasteners—e.g., structural elements that permit the modular housing 102 to be assembled together, such as but not limited to snap together fasteners.

As may be more fully appreciated in FIG. 1C, the first end component 112, the second end component 114 and the midsection component 116 are structured and arranged to receive and hold the flow rate regulators 104 in selected alignment. More specifically, for at least one embodiment the components of the modular housing 102 are structured and arranged with hollowed areas 118 specifically sized to receive and/or over seal the aligned flow rate regulators 104 so as to define a configurable flow path 124 through the CFC 100 as between the inlet port 108 and the outlet port 110.

It will also be understood and appreciated that for at least one embodiment, modular housing 102 is a snap together modular housing. More specifically, as shown first end component 112, second end component 114 and midsection component 116 present corresponding latching pegs 120 and receiving holes 122 that are structured and arranged to firmly couple the components to one another in a leak proof assembly when pressed together with sufficient force.

For at least one embodiment there are at least three (3) latching pegs 120 and at least three (3) receiving holes 122 at about 60° intervals about the mating faces of the of the first end component 112 and the midsection component 116. As the second end component 114 may be substantially identical to the first end component 112, for at least one embodiment, the midsection component may be provided in two forms—one with receiving holes 122 on both opposing surfaces, and a second with latching pegs 120 on one mating surface and receiving holes 122 on the other. Alternatively, the second end component 114 may be essentially the same as the first end component except for the latching pegs 120 being replaced by receiving holes 122 instead.

With respect to the force required for assembly, such force is intended to be that of an average adult person, such as but not limited to 35 pounds of compressive force. Further, it will be understood and appreciated that for at least one embedment the modular housing 102 is not intended to be disassembled after use, rather it is intended to be a single use device.

In other words, the component elements of CFC 100—specifically at least a first end component 112, a second end component, at least one flow rate regulator 104 and at least one midsection component 116 may be provided as a kit in one or more sterile packages for assembly by a user without the requirement of glue, clamps, or other assembly tools. Moreover, an embodiment of CFC 100 may be easily assembled in a clinical or non-clinical setting by an average person.

It will also be understood and appreciated, that although the exemplary CFC 100 as shown in FIGS. 1A-1C has been illustrated with two (2) flow rate regulators 104, it will be understood and appreciated that the modular design of the housing 102 is such that the midsection component 116 may be omitted such that the first end component 112 and the second end component 114 may enclose a single flow rate regulator 104. However, the use of two flow rate regulators 104 has been adopted for the present description so as to permit a greater appreciation for the advantageous configuration options permitted for different flow rates.

In addition, it will be understood and appreciated that additional midsection components 116 and nested flow rate regulators 104 may be attached so as to provide a CFC 100 having from 2, 3, 4 . . . to n flow rate regulators 104 disposed therein.

It is this configurable flow path 124 that advantageously permits the CFC 100 to be quickly and easily adjusted to provide a known flow rate for a given pressure. Moreover, in contrast to the traditional use of flow control tubing as discussed above which relies upon the precise and consistently maintained internal diameter of the flow control tubing over specific lengths, the CFC 100 advantageously provides a low cost precise and configurable flow resistance by changing the geometry of the flow path 124 within CFC 100.

More specifically, the resistance of the present invention, e.g., the CFC 100, is not determined solely by the inner diameter of the flow path, but rather by optional turbulence created by intersecting flow paths presented by at least a subset of the selectable flow pathway segments 106 provided by the flow rate regulators 104.

Moreover, as a striking advantage over the fabrication of long and precise tubing, e.g., flow control tubing, the present invention of the CFC 100 advantageously achieves configurable flow rate control through the use of flow path segments 106 which are cut into flat elements, such as gaskets 126, which serve as the flow rate regulators 104. Indeed, it is to be understood and appreciated that each flow path segment 106 is established with a specific geometry so as to establish for each path segment 106 a pre-determined resistance to flow. As is shown in the accompanying figures and further discussed below, it is the specific physical geometry of a given path segment 106 that may create a resistance to the passage of liquid therethrough—and this resistance to flow achieves the advantageous and predetermined known flow rate, or rather the reduction to flow rate, for a given path segment 106. By selectively combining the path segments 106 of different flow rate regulators 104 the configurable flow path 124 is established with an overall flow rate.

Indeed, as a result of the advantageous use of flow path segments 106 with predetermined resistance to flow, it will be understood and appreciated that the inlet port 108 and the outlet port 110 of CFC 100 need not be coupled to flow control tubing so as to achieve a desired pre-selected flow rate.

As shown, for at least one embodiment, these flow rate regulators 104 are circular, however other geometric cross-sectional shapes, such as but not limited to square, triangle, pentagon, hexagon, star, etc. . . . , may be desired in varying embodiments, and may in some cases even be desired so as to help facilitate the alignment of a selected flow path segment 106 of one flow rate regulator 104 with a selected flow path segment 106 of yet another flow rate regulator 104.

For the present embodiment, employing substantially round flow rate regulators 104, each flow rate regulator 104 has been established with at least one aligner 128, such as one or more grooves or sockets 130, which are structured and arranged to receive corresponding ridges or pegs 132 as provided by the inner surfaces of hollowed areas 118 of first end component 112, second end component 114, and/or midsection component(s) 116, between which the flow rate regulator 104 has been disposed.

It will be understood and appreciated that even for embodiments where the flow rate regulators 104 and the hollowed areas 118 are not substantially circular but alternatively selected to present an alternative geometry, such as but not limited to triangles, pentagons, octagons, etc. . . . , aligners 128 may still be provided so as to further facilitate and ensure that the desired configuration of the selectable flow path segments 106 is maintained.

As will be further discussed below with respect to the accompanying figures, the configurable flow path 124 is established by aligning selectable flow pathway segments 106 provided by the flow rate regulators between the inlet port 108 and the outlet port 110, and for embodiments of CFC 100 incorporating at least two flow rate regulators 104, the at least one midsection component 116 will also provide at least one through port 134 to which the selectable flow pathway segments 106 may be aligned so as to permit the flow of fluid from one flow rate regulator 104 to the next.

For ease of discussion and illustration these one or more passages transversely through the midsection component 116 are termed through port 134, though they may also be referred to as through holes, liquid conduits, pipes, etc. . . . but they are understood and appreciated to be distinctly different from the configurable flow pathway segments provided by each flow rate regulator 104

For at least one embodiment, each flow rate regulator 104 provides at least two distinct flow pathway segments 106. It may be appreciated from FIG. 1D that at least one flow pathway segment 106 is a Fixed-Geometry Passive Check Valve, herein after “FGPCV”, 136, and the other pathway segment 106 is a through hole 138.

It will be understood and appreciated that the FGPCV 136, is specifically a pathway structured and arranged to cause turbulence which reduces the pressure and flow rate when fluid passes in a first direction, herein defined as the first orientation or normal orientation, but does not cause turbulence when the fluid passes in a second, opposite direction, herein defined as the second orientation or reversed orientation. For at least one embodiment the FGPCV 136 is a Tesla valve, also known as a valvular conduit.

For at least one embodiment, the FGPCV 136 provides at least two fluid conduits, e.g., 140 and 142, which are structured and arranged between two access ports 144 and 146 to the flow path segment 106 such that fluid passing in one direction is subjected to turbulence, while fluid passing in the opposite direction is not.

As is shown in FIG. 1D, fluid conduit 140 is essentially a straight path from port 144 to port 146, while fluid conduit 142 turns back towards the port 144 such that fluid passing through fluid conduit 142 encounters fluid passing through fluid conduit 140 in the opposite direction when port 144 is selected as the ingress port for the FGPCV 136. In such a configuration port 146 is the egress for the FGPCV 136. For the purposes of the present invention, this is the normal orientation for FGPCV 136.

When this orientation is reversed so that port 146 is the ingress port and port 144 is the egress port, it will be understood and appreciated that the flow thorough FGPCV 136 is reversed, i.e., this is the reversed orientation. As such, fluid conduit 142 is essentially unused, and even to the extent that some fluid may follow it in reverse, the reunion with fluid conduit 140 at port 144 does not result in sufficient, if even measurable, turbulence, as is induced in the normal orientation.

Moreover, it will be understood and appreciated that exemplary FGPCV 136 having fluid conduits 140 and 142 are but one embodiment for such a FGPCV 136. Indeed, those skilled in the art will understand and appreciate that alternative configurations of multiple fluid conduits, or even just one fluid conduit having baffles, buckets, cups, enlargements, projections or the like may be employed as alternatives or augmentations thereto are within the scope and expectation of a Fixed-Geometry Passive Check Valve as used herein. In other words, it is the ability to impart a greater resistance in one direction and a lesser resistance or none at all in the opposite direction which is inherent to the advantageous configurability of CFC 100.

In other words, the FGPCV 136 is structured and arranged to have a higher pressure drop for the flow in one direction (reverse, then in the other (forward). This difference in flow resistance causes a net directional flow rate in the forward direction in oscillating flows. This efficiency is often expressed in diodicidy Di, being the ratio of the directional resistances.

The flow resistance is defined as the ratio of applied pressure drop to the resulted flow rate, which is analogous to Om's law for electrical resistance, and the FGPCV may be understood with respect to the behavior of a diode—an electrical semiconductor device that essentially acts as a one-way switch for current—allowing easy flow in one direction, but severely limited flow in the opposite. This may be further understood with respect to the following equations.

$$R = \frac{Δ p}{Q}$$

where Δp is me applied pressure difference between the two ends of the conduit, and [symbol] is the flow rate. The diodicity is then:

$$Di = \frac{Rr}{{R}_{f}} .$$

If Di>1, then the conduit in question has diodic behavior.

More simply put, an exemplary embodiment of a flow rate regulator 104 may be understood and appreciated to two selectable flow pathway segments 106—a through hole 138 structured and arranged to provide essentially no resistance to flow rare (0%) and a FGPCV 136 structured and arranged to have a 2% resistance to flow rate when the fluid is not subject to turbulence (fluid passes from port 146 to port 144 through fluid conduit 140) and a 10% resistance to flow rate when the fluid is subject to turbulence) fluid passes from port 144 to 146 through fluid conduits 140 and 142. These reduction to flow rate values are understood and appreciated to be merely exemplary for ease of discussion, and not limitations. It will be appreciated that the resistance to flow rate imposed by each selectable pathway segment 106 is essentially cumulative such that various different, but known, overall flow rates for CFC 100 may be established by selectively aligning the flow rate regulators 104 to establish a desired overall flow path. Because the resistance to flow rate is essentially the same from one instance of a flow pathway segment 106 to the next, it will be understood and appreciated that the actual flow rate through a first instance of a pathway segment 106 may be different from the actual flow rate through a second instance of essentially the same pathway segment 106.

With respect to the above overview, at least one embodiment for a CFC 100 may be summarized as a modular housing 102 providing an inlet port 108 structured and arranged for connection to a first tubing line from the reservoir and an outlet port 110 structured and arranged for connection to a second tubing line to a patient; and a user configurable flow path 124 disposed within the modular housing 102 between the inlet port 108 and the outlet port 110 defined by a plurality of optional pathway segments 106 including at least one fixed-geometry passive check valve 136, each optional pathway segment 106 having a predetermined resistance to flow, a selected subset of the optional pathway segments 106 establishing the selected known flow rate for the liquid passing from the outlet port 110.

Although in a classic implementation, a FGPCV pathway presented as a Tesla valve has very significant flow resistance in one direction and essentially no flow resistance in the opposite second direction, it will be understood and appreciated that for at least one embodiment of CFC 100, the FGPCV 136 is structured and arranged to impart a known flow restriction for a given pressure in either direction—a first flow restriction for a first direction from port 144 to port 146, and a second flow restriction for a second direction from port 146 to port 144, with the first flow restriction greater than the second flow restriction.

As noted above, flow rate regulator 104 also has a selectable flow pathway segment 106 which for at least one embodiment is a through hole 138. In contrast to the FGPCV 136, for at least one embodiment the through hole 138 provides essentially no flow restriction as the fluid is permitted to transfer through the flow rate regulator 104. Those skilled in the art will understand and appreciate that, as noted above, essentially each and every element of a flow path can, and likely does, impart some element to the overall flow rate, but just as with traditional medical tubing when compared to flow control tubing, the element of flow restriction provided by through hole 138 is essentially negligible with respect to the performance of CFC 100.

For yet another embodiment, the selectable flow pathway segment 106 provided as a through hole 138 is optionally structured and arranged to provide a maximum flow rate that is at or slightly below the maximum prescribed flow rate for a given medication that may be used for a particular infusion therapy.

With respect to the exemplary embodiment shown of CFC 100, each flow rate regulator 104 provides at least three different configurable flow rates. With respect to present embodiments of CFC 100 utilizing from 1 to n flow rate regulators 104, it will be understood and appreciated that there are 3ⁿ possible flow rate configurations, though in some cases two different configurations present the same flow rate as one configuration is essentially the same as another.

It should also be understood and appreciated, that although the exemplary flow rate regulator 104 is shown to have a single instance of a FGPCV 136, specifically shown as a tesla valve, for at least one embodiment a plurality of such fixed-geometry passive check valve pathway segments 136 may also be provided on each flow rate regulator 104. However, the advantageous benefit of multiple FGPCV 136 in series may also be easily and advantageously achieved by selectively configuring the alignment of multiple flow rate regulators 104 as shown in FIGS. 1C 1A and 1B.

With two flow rate regulators 104, e.g., 104A and 104B, as shown in FIGs. 1A and 1C, there are essentially nine (9) different flow path configurations, though three (3) are essentially the same—leaving 6 distinct different flow rate configurations. FIG. 2 presents a high-level overview of these 6 different configurable flow path options as may be achieved with two flow rate regulators 104 as described above. As noted, each flow rate regulator 104 has a Through Hole Segment (“TH”) and a Fixed Geometry Passive Check Valve Segment (“FGPCV”) having two orientations, with the reversed orientation being faster than the normal orientation).

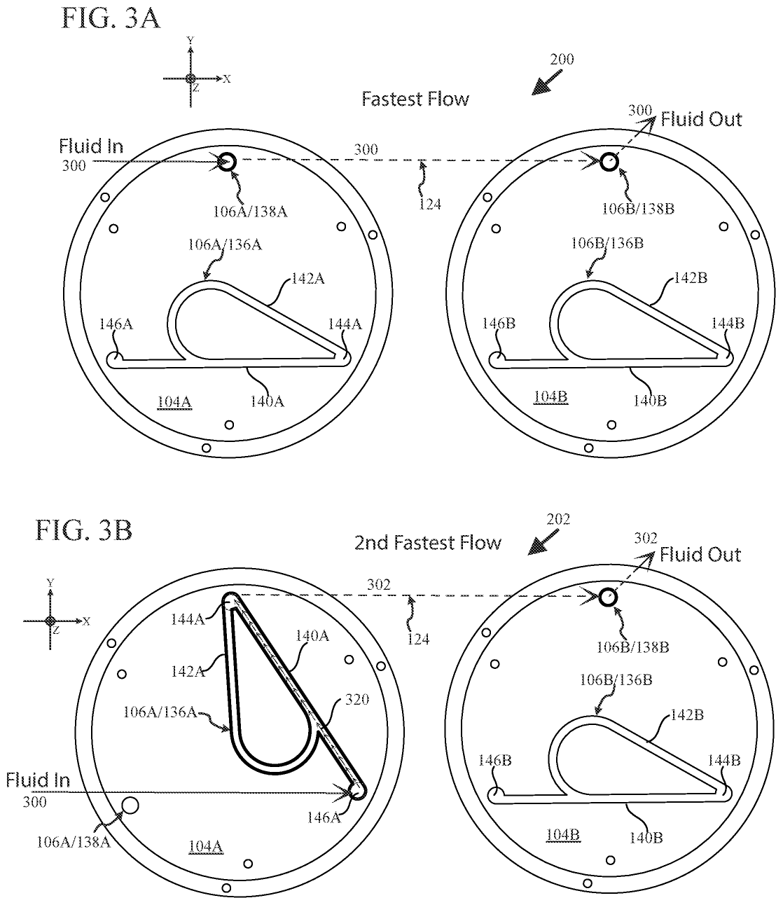

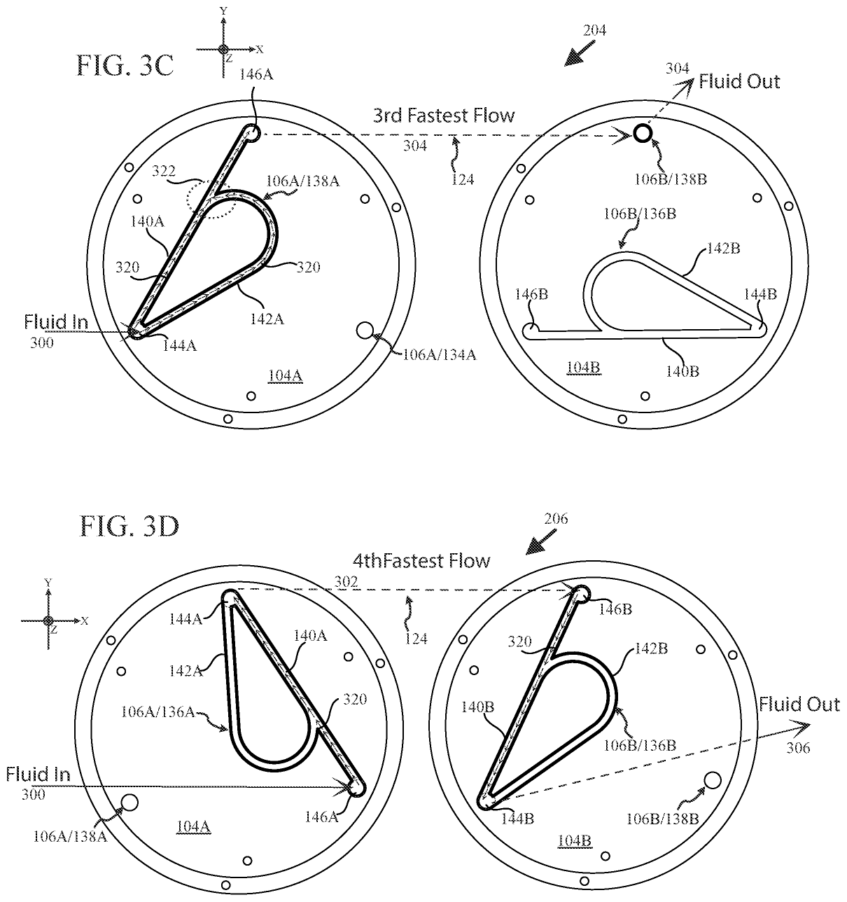

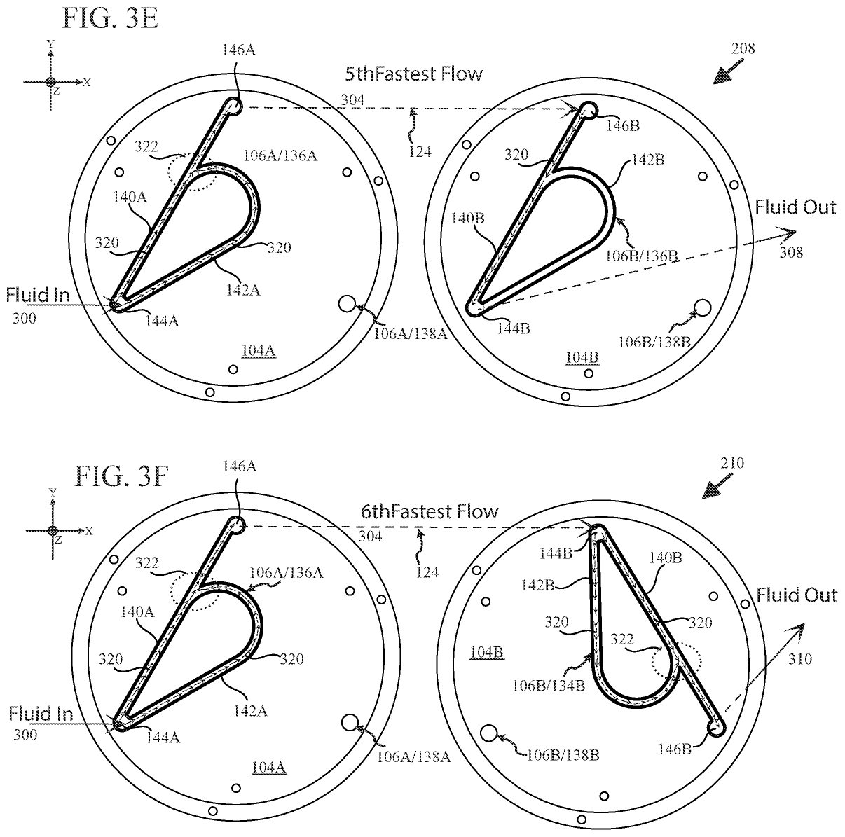

More specifically, FIG. 2 presents these six different configurations side by side for visual comparison and appreciation of the differences with FIGS. 3A-3F providing enlarged depictions of each configuration. In FIG. 2, the selected pathway segments 106 have been rendered with thickened lines for further distinction.

Moreover, in FIG. 2, option 200 shows alignment configuration 1: TH+TH=fastest flow; option 202 shows alignment configuration 2: FGPCV (reversed)+TH=2^{nd} fastest flow; option 204 shows alignment configuration 3: FGPCV (normal)+TH=3rd fastest flow; option 206 shows alignment configuration 4: FGPCV (reversed)+FGPCV (reversed)=4^{th} fastest flow; option 208 shows alignment configuration 5: FGPCV (normal)+FGPCV (reversed)=5^{th} fastest flow; and option 210 shows alignment configuration 6: FGPCV (normal)+FGPCV (normal)=6^{th} fastest flow. For each configuration 200-210, the configured flow path 124 is shown with a series of arrows 212.

FIGS. 3A through 3F are provided to further illustrate these alignment configuration options. Of course, it will be understood and appreciated that the scale of the illustrations has been adjusted for ease of illustration and discussion, and is not intended to suggest or imply a limitation of size and scale for the selectable flow pathway segments 106.

As in FIG. 2, FIGS. 3A through 3F are arranged to show decreasing flow rates from the fastest configurable flow rate, shown in FIG. 3A, to the slowest configurable flow rate, shown in FIG. 3F. For ease of illustration and discussion, FIGS. 3A-3F have been rendered essentially as plane views of the first flow regulator 104A and the second flow regulator 104B without the first end component 112 or second end component 114 of CFC 100 to more clearly depict their optional alignments to provide configurable flow path 124 as between the inlet port 108 and outlet port 110, which are not shown in FIGS. 3A-3F. With respect to FIGS. 3A through 3F the suffix of “A” or “B” is added to appropriate element numbers to indicate the element as being discussed with respect to the first flow rate regulator 104A or the second flow rate regulator 104B—the first flow rate regulator shown 104A on the left, and the second flow rate regulator 104B shown on the right. Further, throughout FIGS. 3A through 3F, the original flow rate of fluid being provided to CFC 100 is understood and appreciated to be the same fluid flow rate 300

More specifically, as shown in FIG. 3A, the first flow regulator 104A is shown configured to present the selectable flow pathway segment 106A of the through hole 138A to receive incoming fluid, shown by an arrow at the initial flow rate 300. Similarly, the second flow regulator 104B is shown configured to present the selectable flow pathway segment 106B of the through hole 138B to receive incoming fluid exiting from flow rate regulator 104A, shown by dashed arrow, with the exiting fluid directed out of CFC 100 through the outlet port 110 (shown in FIGS. 1A and 1B). As this configurable flow path 124 is for through hole 138A to through hole 138B it is the fastest flow/least restricted flow, and the existing flow rate is therefore shown to be 300, substantially the same as the original incoming flow rate also shown as 300.

As shown in FIG. 3B, the first flow rate regulator 104A has been rotated to align port 144A of the FGPCV 136A with the through hole 138B of the second flow rate regulator 104B. As such, incoming fluid, shown by an arrow at the initial flow rate 300, is received by port 146A of first flow rate regulator 104A, and follows fluid conduit 140A as shown by small arrows to port 144A where it exists from the first flow rate regulator 104A and enters the second flow rate regulator 104B, shown by arrow as flow rate 302, the flow rate having been reduced by the passage through conduit 140A. Because this is the second orientation, aka reversed orientation for FGPCV 136A, there is little, if any flow through conduit 142A, which is indicated by the lack of small arrows 320

As shown, second flow rate regulator 104B has been configured in an orientation to receive the fluid at flow rate 302 into through hole 138B, with the exiting fluid directed out of CFC 100 through the outlet port 110 (shown in FIGS. 1A and 1B). As this configurable flow path 124 is for conduit 140A to through hole 138B it is the second fastest flow/second least restricted flow, the exiting flow rate is essentially the flow rate imparted by fluid conduit 140A, shown as flow rate 302.

In FIG. 3C, the first flow rate regulator 104A has been rotated to align port 146A of the FGPCV 136A with the through hole 138B of the second flow rate regulator 104B. As such, incoming fluid, shown by an arrow at the initial flow rate 300, is received by port 144A of flow rate regulator 104A, and follows fluid conduits 140A and 142A as shown by small arrows 320 in both conduits 140A and 142A, experiencing intended turbulence 322, to port 146A where it exists from the first flow rate regulator 104A and enters the second flow rate regulator 104B, shown by arrow as flow rate 304, the flow rate having been reduced by the turbulence imposed by passage through both fluid conduit 140A and fluid conduit 142A.

As shown, second flow rate regulator 104B has been configured in an orientation to receive the fluid at flow rate 306 into through hole 138B, with the exiting fluid directed out of CFC 100 through the outlet port 110 (shown in FIGS. 1A and 1B). As this configurable flow path 124 is for fluid conduits 140A and 142A (experiencing intended turbulence) to through hole 138B it is the third fastest flow/third least restricted flow, the exiting flow rate is essentially the flow rate imparted by FGPCV 136A with both fluid conduits 140A and 142A and their resulting turbulence, shown as flow rate 304.

In FIG. 3D, the first flow rate regulator 104A has been rotated to align port 144A of the FGPCV 136A with port 146B of the of the FGPCV 136B of the second flow rate regulator 104B. As such, incoming fluid, shown by an arrow at the initial flow rate 300, is received by port 146A of first flow rate regulator 104A, and follows fluid conduit 140A as shown by small arrows 320 to port 144A where it exits from the first flow rate regulator 104A and enters the second flow rate regulator 104B, shown by an arrow as flow rate 302.

As shown, the second flow rate regulator 104B has been configured in an orientation to receive the fluid at the reduced flow rate 302 into port 146B, the fluid then following fluid conduit 140B as shown by small arrows 320 to port 144B where it exists from the second flow rate regulator 104B, at the yet further reduced flow rate 306. As this configurable flow path 124 is for conduit 140A to conduit 140B it is the fourth fastest flow/fourth least restricted flow, the exiting flow rate 306 being the result of both fluid conduits 140A and 140B, shown as exiting flow rate 306.

In FIG. 3E, the first flow rate regulator 104A has been rotated to align port 146A of the FGPCV 136A with port 146B of the of the FGPCV 136B of the second flow rate regulator 104B. As such, incoming fluid, shown by an arrow at the initial flow rate 300, is received by port 144A of flow rate regulator 104A, and follows fluid conduits 140A and 142A as shown by small arrows 320 in both conduits 140A and 142A, experiencing intended turbulence 322, to port 146A where it exists from the first flow rate regulator 104A and enters the second flow rate regulator 104B, shown by arrow as flow rate 304, the flow rate having been reduced by the turbulence imposed by passage through both fluid conduit 140A and fluid conduit 142A.

As shown, the second flow rate regulator 104B has been configured in an orientation to receive the fluid at the reduced flow rate 304 into port 146B, the fluid then following fluid conduit 140B as shown by small arrows 320 to port 144B where it exists from the second flow rate regulator 104B, at the yet further reduced flow rate 308. As this configurable flow path 124 is for fluid conduits 140A and 142A (experiencing intended turbulence) to conduit 140B it is the fifth fastest flow/fifth least restricted flow, the exiting flow rate 308 being the result of the flow rate imparted by FGPCV 136A with both fluid conduits 140A and 142A and their resulting turbulence and conduit 140B, shown as exiting flow rate 308.

In FIG. 3F, the first flow rate regulator 104A has been rotated to align port 146A of the FGPCV 136A with port 144B of the of the FGPCV 146B of the second flow rate regulator 104B. As such, incoming fluid, shown by an arrow at the initial flow rate 300, is received by port 144A of flow rate regulator 104A, and follows fluid conduits 140A and 142A as shown by small arrows 320 in both conduits 140A and 142A, experiencing intended turbulence 322, to port 146A where it exists from the first flow rate regulator 104A and enters the second flow rate regulator 104B, shown by arrow as flow rate 304, the flow rate having been reduced by the turbulence imposed by passage through both fluid conduit 140A and fluid conduit 142A.

As shown, the second flow rate regulator 104B has been configured in an orientation to receive the fluid at the reduced flow rate 304 into port 144B, and follows fluid conduits 140B and 142B as shown by small arrows 320 in both conduits 140B and 142B, experiencing intended turbulence 322, to port 146B where it exists from the second flow rate regulator 104B, at the yet further reduced flow rate 310.

As this configurable flow path 124 is for fluid conduits 140A and 142A (experiencing intended turbulence) and conduits 140B and 142B (experiencing intended turbulence), it is the sixth fastest flow/most restricted flow, the exiting flow rate 210 being the result of the flow rate imparted by FGPCV 136A with both fluid conduits 140A and 142A and their resulting turbulence and by FGPCV 136B with both fluid conduits 140B and 142B and their resulting turbulence, shown as exiting flow rate 310.

Moreover, with respect to the above description and presentation of figures, it will be understood and appreciated that for at least one embodiment, CFC 100 is summarized as consisting of: a modular housing 102 providing an inlet port 108 structured and arranged for connection to a first tubing line from the reservoir and an outlet port 110 structured and arranged for connection to a second tubing line to the patient; and a configurable flow path 124 provided by at least a first flow rate regulator 104 and at least a second flow rate regulator 104, each flow rate regulator 104 providing selectable flow pathway segments 106 including at least one fixed-geometry passive check valve and at least one through hole with each pathway segment having a predetermined resistance to flow, the selectable alignment of the first flow rate regulator 104A to the second flow rate regulator 104B aligning at least two flow pathway segments 106 between the inlet port 108 and the outlet port 110 to provide the selected known flow rate for the liquid passing from the outlet port 110.

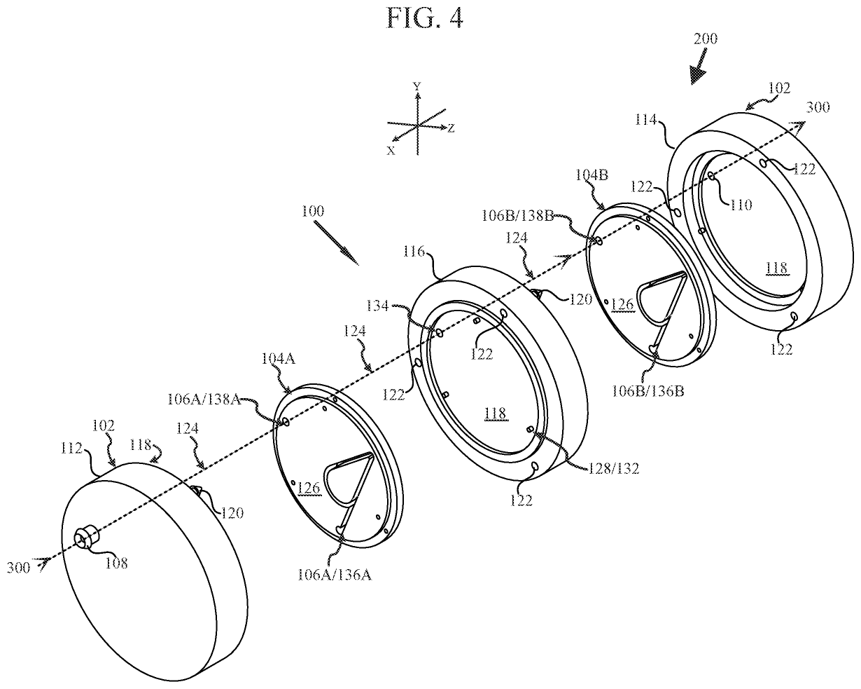

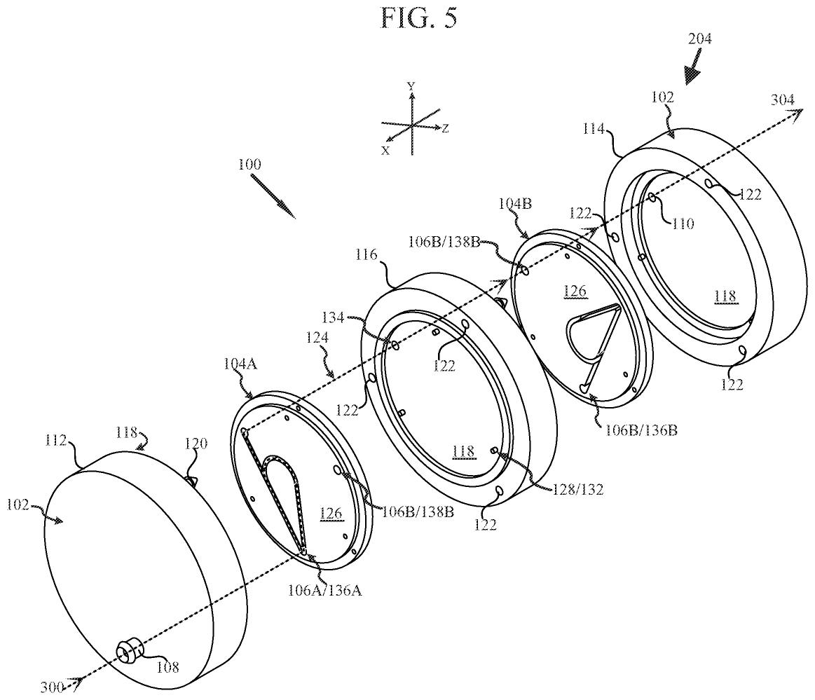

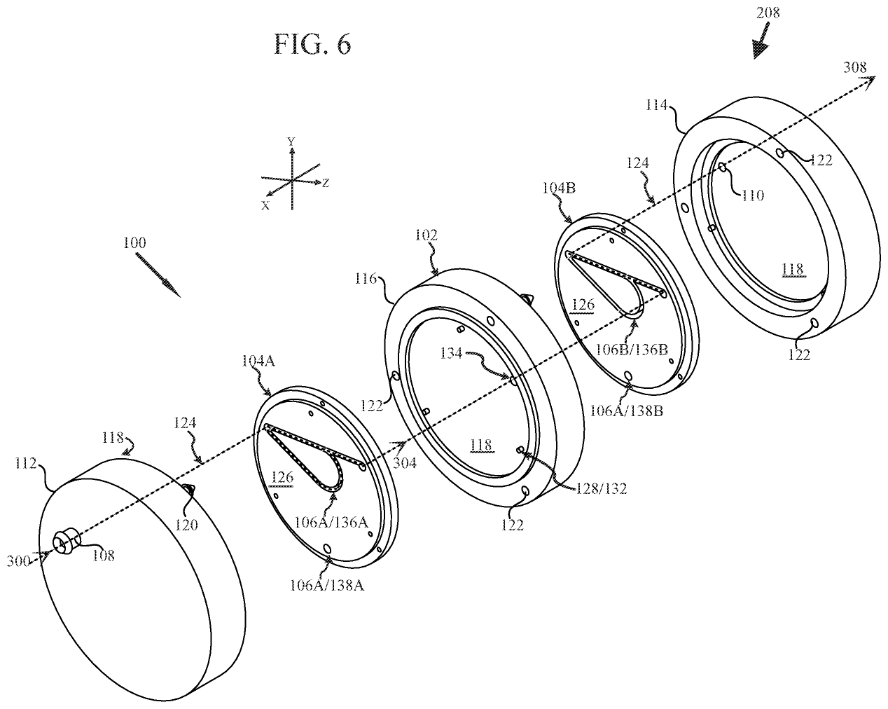

To further appreciate the optional configurations for configurable flow path 124 as presented in FIG. 2 and in greater detail in FIGS. 3A through 3F, FIG. 4 is an exploded perspective view of a CFC 100 in configuration 200—specifically the TH 138A to TH 138B of flow rate regulators 104A and 104B as between the inlet port 108 and the outlet port 110 of housing 102.

Similarly, FIG. 5 is an exploded perspective view of a CFC 100 in configuration 204—specifically the FGPCV 136A normal to TH 138B of flow rate regulators 104A and 104B as between the inlet port 108 and the outlet port 110 of housing 102.

And further, FIG. 6 is an exploded perspective view of a CFC 100 in configuration 208—specifically the FGPCV 136A normal to FGPCV 136A reversed of flow rate regulators 104A and 104B as between the inlet port 108 and the outlet port 110 of housing 102.

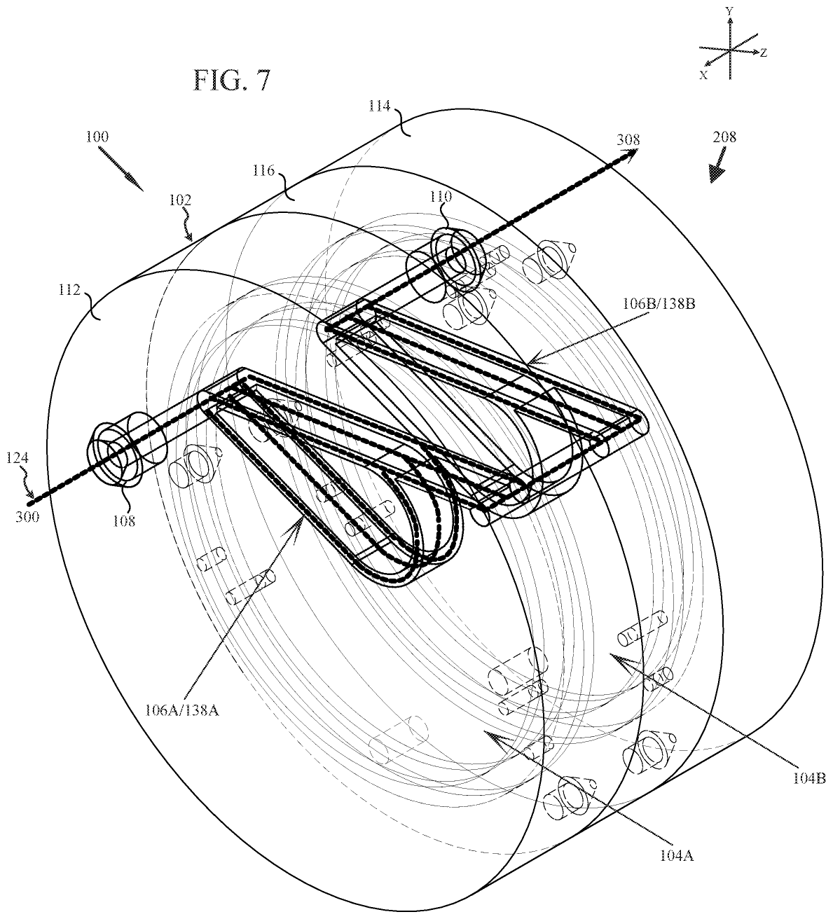

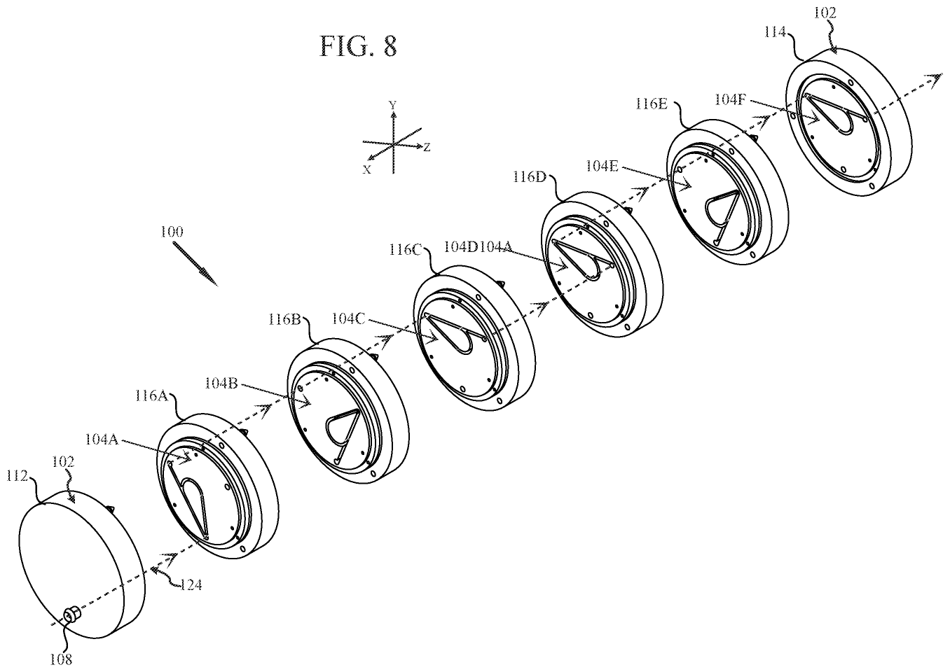

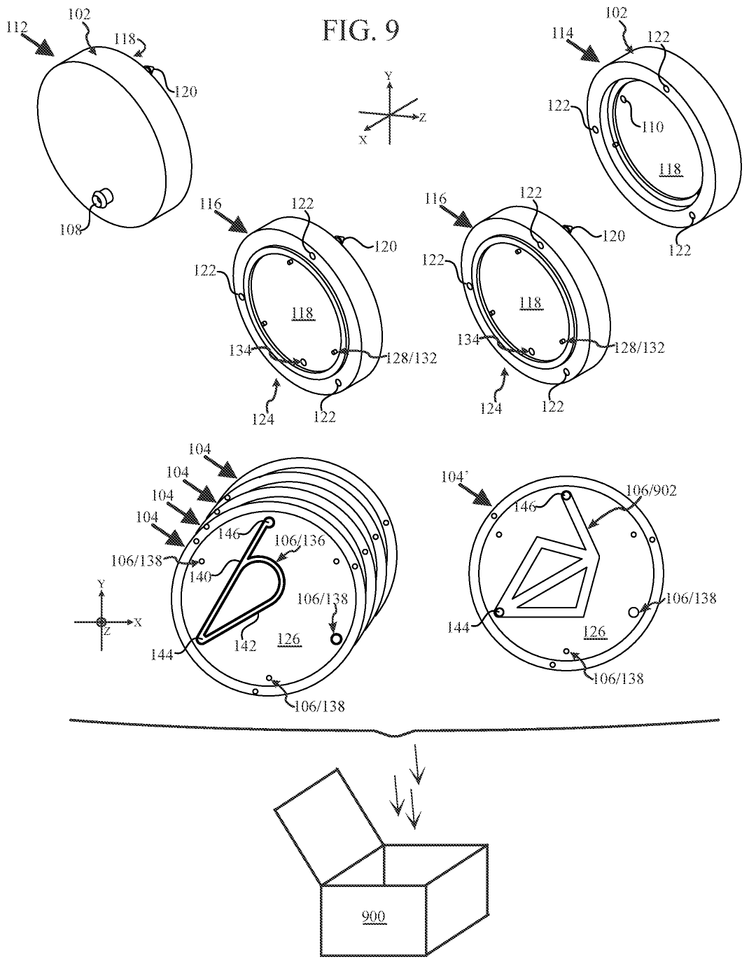

To further appreciate the actual assemblage of CFC 100, FIG. 7 provides a perspective view with dotted elements illustrating the internal components of the CFC 100 in configuration 208 shown in FIG. 6 for configuration 206. In FIG. 7, FGPCV 136A and FGPCV 136B have been rendered in heavy dashed lines to accentuate their presence within the CFC 100. The configurable pathway 124 is shown in heavy dash passing thorough both conduits of FGPCV 136A, but only the straight conduit of FGPCV 136B.

FIG. 8 illustrates yet another embodiment of CFC 100′, in this case one in which there are six (6) instances flow rate regulator 104, e.g., flow rate regulators 104A, 104B, 104C, 104D, 104E, and 104F. For ease of illustration each flow rate regulator 104A, 104B, 104C, 104D and 104E is shown now disposed in the hollowed area of its adjacent midsection component 116A, 116B, 116C, 116D and 116E. Flow rate regulator 104F is shown disposed in the hollowed area of second end component 116. The configurable flow path 124 established through CFC 100 is also shown.

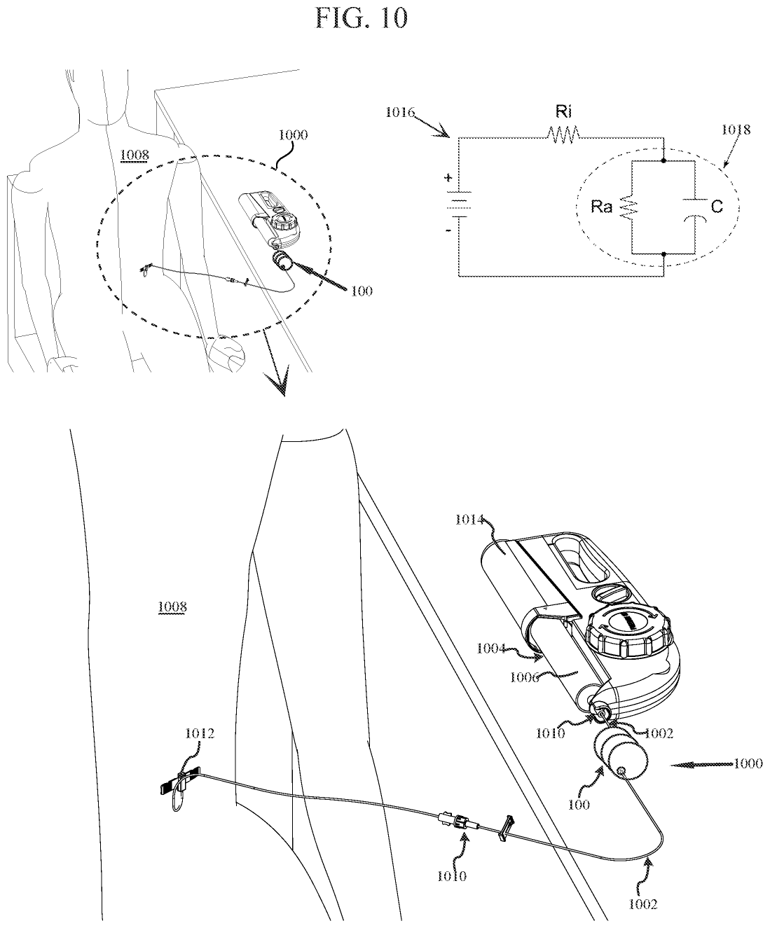

To expand on the notion from above of easy user assembly, in or out of a medical environment, for at least one alternative embodiment CFC 100 is provided as a kit which may be selectively assembled by a user/operator, to establish a selected flow rate for a given pressure rate for an infusion therapy. FIG. 9 conceptually illustrates such an embodiment for a kit 900.

As shown in FIG. 9, for at least one embodiment, kit 900 includes one first end component 112, one second end component 114, a plurality of midsection components 116, and a plurality of flow regulators 104.

With respect the various configurations exemplified in the above figures, it will be understood and appreciated that the flow rate regulator 104A and flow rate regulator 104B are understood and appreciated to be substantially identical. Such an embodiment may be desirable for the simplification of fabrication, e.g., a plurality of flow rate regulators 104 may be fabricated from the same template and as all are essentially identical there is no requirement for distinct labeling.

However, it is understood and appreciated that for at least one embodiment, CFC 100 may incorporate at least two flow rate regulators 104 that are not substantially identical. Indeed, for at least one embodiment, an alternative flow rate regulator may be provided having a FGPCV provided therein which provides a different flow rate in either or both the normal and reversed orientations from FGPCV 136. To exemplify this, kit 900 may also include one or more alternative flow rate regulators 104′ wherein at least one selectable flow pathway segment 106 is an alternative FGPCV 902 which presents a fixed geometry different from that of FGPCV 136. To accentuate the issue of resistance to flow in one direction, but not in the other, Alternative FGPCV 902 may also incorporate baffles, buckets, cups, enlargements, projections or the like—not presently shown due to sale and for ease of illustration and discussion. Of course, it will be understood and appreciated that the one or more alternative flow rate regulators 104′ may simply provide an alternative tesla valve as FGPCV 902—one that is larger, or smaller, or otherwise configurated differently so as to provide an alternative resistance to flow from that which is predetermined and intended for one or more instances of FGPCV 136, which may also be provided.

Such a kit 900 may be summarized as a kit for a CFC 100 for providing a selected known rate for a given pressure rate for the delivery of a liquid from a reservoir to a patient, including: a segmented housing 102 providing an inlet port 108 structured and arranged for connection to a first tubing line from the reservoir and an outlet port 110 structured and arranged for connection to a second tubing line to the patient; and a plurality of flow rate regulators 104 to be disposed by a user within the segmented housing 102, each flow rate regulator 104 providing at least one selectable flow path segment 106 with at least one selectable flow path segment 106 being a fixed-geometry passive check valve 136; wherein a user selectable alignment of at least a first flow rate regulator 104 to a second flow rate regulator 104 aligning at least two flow pathway segments 106 between the inlet port 108 and the outlet port 110 as a configurable flow path 124 to provide the selected known flow rate for the liquid passing from the outlet port 110.

With respect to the introduction above and the description of an embodiment of CFC 100 being used to adaptatively provide a configurable flow rate for an infusion system, FIG. 10 conceptually illustrates such a system 1000.

More specifically, a CFC 100 as shown and described above with respect to FIGS. 1A-1D, is shown disposed in a tubing system 1002 between a reservoir 1004 of liquid 1006, and a patient 1008. The tubing system 1002 may be comprised of normal medical tubing and fitted with various connectors such as luers 1010 which facilitate connection to the reservoir 1004 and a needle set 1012 that is disposed in the patient 1008 set to receive the infusion therapy.

For at least one embodiment, the through hole 138 segments of the configurable flow path 124 (shown in FIGS. 1A-1D) of CFC 100, such as the through hole 138 segments, may be pre-established so as to limit the flow rate to the maximum prescribed flow rate for the liquid 1006, and as so configured, provide an assurance that CFC 100 will not permit a flow rate above the recommended flow rate. For yet other embodiments, the through hole 138 segments 106 of the configurable flow path 124 of CFC 100 are true through holes imparting no significant restriction of flow, such that the assurance of proper flow rate is established by the configuration of the at least one FGPCV 136 (shown in FIGS. 1A-1D) within the CFC 100.

As shown, the reservoir 1004 is provided by a syringe that is disposed in a pump 1014. For at least one embodiment the pump 1014 is a constant pressure pump 1014 such as the Freedom60® Syringe Infusion Pump as provided by RMS Medical Products of Chester, N.Y., DBA Koru Medical Systems. Constant pressure systems, such as constant pressure pump 1014, when combined with CFC 100 may be highly advantageous in preventing unintended and/or unsafe rates of administration of the liquid 1006 to the patient 1008.

With a constant flow rate system, the pressure is increased in response to any flow restriction no matter if such a restriction is the build-up of pressure in the patient's tissues or an element of the delivery system. This can result in an administration of the liquid at a unsafe pressure. As such, the patient may suffer a wide range of symptoms, including, but not limited to, vein collapse, anaphylaxis, overdose, histamine reactions, morbidity, and mortality.

In sharp contrast, with a constant pressure rate system, such as constant pressure pump 1014, if there is a pinch in the tubing, blockage in the infusion system or blockage in the patient's body (such as by saturation of the tissues), results in resistance to the flow and affects the flow rate, not the pressure, i.e., the flow rate decreases as the pressure increases. A constant pressure system may be compared to a theoretical model of an electrical system 1016 shown in FIG. 10.

For electrical system 1016, as resistance increases 1018, the current will immediately and proportionally decrease. A constant pressure infusion system produces this same result: if the resistance to flow increases, the system will immediately adjust by lowering the flow rate. This insures—by design—that a patient 1008 can never be exposed to a critically high pressure of liquid 1006.

Moreover, as CFC 100 may establish an upper boundary for flow rate of a liquid 1006 from a reservoir 1004 at or below a pre-defined flow rate, embodiments of CFC 100 are suitable for infusion treatments with constant pressure systems. Additional advantages may be provided when embodiments of CFC 100 are combined with constant pressure pump 1014 such as the Freedom60®.

Having described several physical embodiments of CFC 100, other embodiments relating to a method 1100 of using CFC 100 will now be discussed with respect to FIG. 9. It will be understood and appreciated that the described method 1100 need not be performed in the order in which it is herein described, but that this is merely exemplary of one method of using CFC 100.

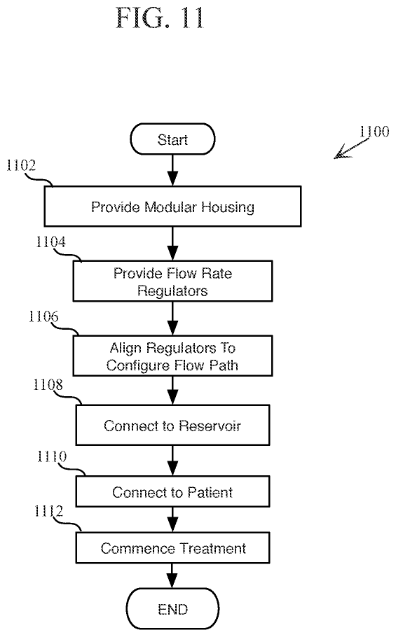

FIG. 11 conceptually illustrates a high-level flow diagram depicting at least one method 1100 for providing and/or using a CFC 100 as shown in FIGS. 1-8. Moreover, method 1100 generally commences with providing a modular housing 102 having an inlet port 108 structured and arranged for connection to a first tubing line from a reservoir of liquid medicant, and an outlet port 110 structured and arranged for connection to a second tubing line to the patient, block 1102.

Method 1100 continues by providing at least two flow rate regulators 104 to be disposed within the modular housing, each rate regulator 104 providing at least one selectable flow pathway segment with at least one selectable flow pathway segment being a fixed-geometry passive check valve 136, block 1104.

The user then selectively aligns the first flow rate regulator 104A to a second flow rate regulator 104B and disposes them within the modular hosing to align and dispose at least two flow pathway segments 106 between the inlet port 108 and the outlet port 110 as a configurable flow path 124 to provide the selected known flow rate for the liquid passing from the outlet port 110, block 1106.

The user or operator then engages tubing; from the inlet port 108 to the liquid reservoir; block 1108, and engages tubing from the outlet port 110 to a needle set for the delivery of the liquid to the patient, block 1110.

With the CFC 100 now assembled and disposed within the infusion system to properly regulate the flow of liquid medicant from the reservoir to the patient, the infusion treatment may commence, block 1112.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A configurable flow controller for providing a selected known flow rate for a given pressure for the delivery of a liquid from a reservoir to a patient, comprising:
a modular housing providing an inlet port structured and arranged for connection to a first tubing line from the reservoir and an outlet port structured and arranged for connection to a second tubing line to the patient; and
a user configurable flow path disposed within the modular housing between the inlet port and the outlet port defined by a plurality of optional pathway segments including at least one fixed-geometry passive check valve, each optional pathway segment having a predetermined resistance to flow, a selected subset of the optional pathway segments establishing the selected known flow rate for the liquid passing from the outlet port.

2. The configurable flow controller of claim 1, wherein the fixed-geometry passive check valve is a Tesla valve.

3. The configurable flow controller of claim 1, wherein the configurable flow path is provided by a plurality of flow rate regulators, each providing at least one selectable flow pathway segment with at least one selectable flow pathway segment being the fixed-geometry passive check valve.

4. The configurable flow controller of claim 3, wherein at least one flow rate regulator provides a selectable flow pathway segment as a through hole.

5. The configurable flow controller of claim 4, wherein the through hole is structured and arranged to permit the unimpeded flow of fluid through the at least one flow rate regulator.

6. A configurable flow controller for providing a selected known flow rate for a given pressure for the delivery of a liquid from a reservoir to a patient, comprising:
a modular housing providing an inlet port structured and arranged for connection to a first tubing line from the reservoir and an outlet port structured and arranged for connection to a second tubing line to the patient; and
a configurable flow path provided by at least a first flow rate regulator and at least a second flow rate regulator, each flow rate regulator providing selectable flow pathway segments including at least one fixed-geometry passive check valve and at least one through hole with each pathway segment having a predetermined resistance to flow, the selectable alignment of the first flow rate regulator to the second flow rate regulator aligning at least two flow pathway segments between the inlet port and the outlet port to provide the selected known flow rate for the liquid passing from the outlet port.

7. The configurable flow controller of claim 6, wherein the fixed-geometry passive check valve is a Tesla valve.

8. The configurable flow controller of claim 6, wherein the fixed-geometry passive check valve has a first flow rate for a first orientation and a second flow rate for a second orientation, the first flow rate different from the second flow rate.

9. The configurable flow controller of claim 6, wherein the first flow rate regulator and the second flow rate regulator are about identical.

10. The configurable flow controller of claim 6, wherein the first flow rate regulator provides a first fixed-geometry passive check valve having a first flow rate and the second flow rate regulator provides a second fixed-geometry passive check valve having a second flow rate different from the first flow rate.

11. The configurable flow controller of claim 6, wherein the modular housing is a snap-together housing.

12. The configurable flow controller of claim 6, wherein the first flow rate regulator and the second flow rate regulator are each gaskets having the selectable flow pathway segments defined therein, each gasket having at least one aligner structured and arranged to permit pre-determined alignments as between the gaskets.

13. The configurable flow controller of claim 12, wherein the at least one aligner is provided by corresponding predefined placement of mating ridges and grooves.

14. The configurable flow controller of claim 12, wherein the at least one aligner is provided by corresponding predefined placement of mating pegs and sockets.

15. The configurable flow controller of claim 6, wherein the through hole is structured and arranged to permit the unimpeded flow of fluid through the at least one flow rate regulator.

16. A configurable flow controller for providing a selected known flow rate for a given pressure for the delivery of a liquid from a reservoir to a patient, comprising:
a segmented housing providing an inlet port structured and arranged for connection to a first tubing line from the reservoir and an outlet port structured and arranged for connection to a second tubing line to the patient; and
at least two flow rate regulators disposed within the segmented housing, each flow rate regulator providing at least one selectable flow pathway segment with at least one selectable flow pathway segment being a fixed-geometry passive check valve;
wherein a user selectable alignment of a first flow rate regulator to a second flow rate regulator aligning at least two flow pathway segments between the inlet port and the outlet port provides the selected known flow rate for the liquid passing from the outlet port.

17. The configurable flow controller of claim 16, wherein the fixed-geometry passive check valve path is a Tesla valve.

18. The configurable flow controller of claim 16, wherein at least one flow rate regulator provides a selectable flow pathway segment as a through hole.

19. The configurable flow controller of claim 18, wherein the through hole is structured and arranged to permit the unimpeded flow of fluid through the at least one flow rate regulator.

20. The configurable flow controller of claim 16, wherein the first flow rate regulator and the second flow rate regulator are about identical.

21. The configurable flow controller of claim 16, wherein the modular housing is a snap-together housing.

22. The configurable flow controller of claim 16, wherein the first flow rate regulator and the second flow rate regulator are each gaskets having the selectable flow pathway segments defined therein, each gasket having at least one aligner structured and arranged to permit pre-determined alignments as between the gaskets.

23. The configurable flow controller of claim 22, wherein the at least one aligner is a provided by corresponding predefined placement of mating ridges and grooves.

24. The configurable flow controller of claim 22, wherein the at least one aligner is a provided by corresponding predefined placement of mating pegs and sockets.

25. A kit for a configurable flow controller for providing a selected known rate for a given pressure rate for the delivery of a liquid from a reservoir to a patient, comprising:
a segmented housing providing an inlet port structured and arranged for connection to a first tubing line from the reservoir and an outlet port structured and arranged for connection to a second tubing line to the patient; and
a plurality of flow rate regulators to be disposed by a user within the segmented housing, each flow rate regulator providing at least one selectable flow path segment with at least one selectable flow path segment being a fixed-geometry passive check valve;
wherein a user selectable alignment of at least a first flow rate regulator to a second flow rate regulator aligns at least two flow pathway segments between the inlet port and the outlet port as a configurable flow path to provide the selected known flow rate for the liquid passing from the outlet port.

26. The kit of claim 25, wherein the fixed-geometry passive check valve path is a Tesla valve.

27. The kit of claim 25, wherein at least one flow rate regulator provides a selectable flow pathway segment as a through hole.

28. The kit of claim 27, wherein the through hole is structured and arranged to permit the unimpeded flow of fluid through the at least one flow rate regulator.

29. The kit of claim 25, wherein the first flow rate regulator and the second flow rate regulator are about identical.

30. The kit of claim 25, wherein the modular housing is a snap-together housing.

31. The kit of claim 25, wherein the first flow rate regulator and the second flow rate regulator are each gaskets having the selectable flow pathway segments defined therein, each gasket having at least one aligner structured and arranged to permit pre-determined alignments as between the gaskets.

32. The kit of claim 31, wherein the at least one aligner is a provided by corresponding predefined placement of mating ridges and grooves.

33. The kit of claim 31, wherein the at least one aligner is a provided by corresponding predefined placement of mating pegs and sockets.

34. The kit of claim 25, further including a needle set comprising one or more needles and tubing.

35. A method for using a configurable flow controller for providing a selected known flow rate for a given pressure for the delivery of a liquid from a reservoir to a patient, comprising:
providing a modular housing having an inlet port structured and arranged for connection to a first tubing line from the reservoir and an outlet port structured and arranged for connection to a second tubing line to the patient; and
providing at least two flow rate regulators to be disposed within the modular housing, each flow rate regulator providing at least one selectable flow pathway segment with at least one selectable flow pathway segment being a fixed-geometry passive check valve;
selectively aligning a first flow rate regulator to a second flow rate regulator and disposing them within the modular housing to align and dispose at least two flow pathway segments between the inlet port and the outlet port as a configurable flow path to provide the selected known flow rate for the liquid passing from the outlet port;
engaging tubing from the inlet port to the liquid reservoir; and
engaging tubing from the outlet port to a needle set for the delivery of the liquid to the patient.

36. The method of claim 35, wherein the fixed-geometry passive check valve path is a Tesla valve.

37. The method of claim 35, wherein at least one flow rate regulator provides a selectable flow pathway segment as a through hole.

38. The method of claim 37, wherein the through hole is structured and arranged to permit the unimpeded flow of fluid through the at least one flow rate regulator.

39. The method of claim 35, wherein rein the first flow rate regulator and the second flow rate regulator are about identical.

40. The method of claim 35, wherein the modular housing is a snap-together housing.

41. The method of claim 35, wherein the first flow rate regulator and the second flow rate regulator are each gaskets having the selectable flow pathway segments defined therein, each gasket having at least one aligner structured and arranged to permit pre-determined alignments as between the gaskets.

42. The method of claim 41, wherein the at least one aligner is a provided by corresponding predefined placement of mating ridges and grooves.

43. The method of claim 41, wherein the at least one aligner is a provided by corresponding predefined placement of mating pegs and sockets.
